(19) United States
(12) Patent Application Publication
(10) Pub. No.: US 2008/0169073 A1
(43) Pub. Date: Jul. 17, 2008

(54) PITCH AND STICKIES CONTROL IN PULP AND PAPERMAKING PROCESSES

(75) Inventors: Zu-Feng Xu, Shanghai (CN); William S. Carey, Wallingford, PA (US)

Correspondence Address: HERCULES INCORPORATED, 1313 N. MARKET STREET, WILMINGTON, DE, 19894-0001, US

(73) Assignee: Hercules Inc., Wilmington, DE (US)

(21) Appl. No.: 11/934,836

(22) Filed: Nov. 5, 2007

Related U.S. Application Data
(60) Provisional application No. 60/856,996, filed on Nov. 6, 2006.

(51) Int. Cl. D21F 1/32; C12N 9/00

(52) U.S. Cl. 162/199; 435/183

(57) ABSTRACT

Methods for inhibiting the depositions of organic contaminants from pulp in pulp and papermaking systems are disclosed. A combination of an enzyme and a nonionic polymeric detackifier are added to the pulp or applied to deposition-prone process equipment surfaces of a pulp and papermaking system.

This application claims the benefit of U.S. Provisional Application No. 60/856,996, filed Nov. 6, 2006.

### FIELD OF THE INVENTION

The present invention relates to methods for inhibiting the deposition of organic contaminants in pulp and papermaking systems.

### BACKGROUND OF THE INVENTION

The deposition of organic contaminants (i.e., pitch and stickies) on surfaces in the papermaking process is well known to be detrimental to both product quality and the efficiency of the papermaking process. Some contaminating components occur naturally in wood and are released during various pulping and papermaking processes. Two specific manifestations of this problem are referred to as pitch (primarily natural resins) and stickies (adhesives or coatings from recycled paper). Pitch and stickies have the potential to cause problems with deposition, quality, and efficiency in the process as mentioned above.

The term “pitch” can be used to refer to deposits composed of organic constituents which may originate from these natural resins, their salts, as well as coating binders, sizing agents, and defoaming chemicals which may be found in the pulp. In addition, pitch frequently contains inorganic components such as calcium carbonate, talc, clays, titanium and related materials.

“Stickies” is a term that has been increasingly used to describe deposits that occur in the systems using recycled fiber. These deposits often contain the same materials found in “pitch” deposits in addition to adhesives, hot melts, waxes, and inks.

The deposition of organic contaminants, such as pitch and stickies, can be detrimental to the efficiency of a pulp or paper mill causing both reduced quality and reduced operating efficiency. Organic contaminants can deposit on process equipment in papermaking systems resulting in operational difficulties in the systems. The deposition of organic contaminants on consistency regulators and other instrument probes can render these components useless. Deposits on screens can reduce throughput and upset operation of the system. This deposition can occur not only on metal surfaces in the system, but also on plastic and synthetic surfaces such as machine wires, felts, foils, Uhle boxes and head box components.

Historically, the subsets of the organic deposit problems, “pitch” and “stickies”, have manifested themselves separately, differently and have been treated distinctly and separately. From a physical standpoint, “pitch” deposits have usually formed from microscopic particles of adhesive material (natural or man-made) in the stock which accumulate on papermaking or pulping equipment. These deposits can readily be found on stock chest walls, paper machine foils, Uhle boxes, paper machine wires, wet press felts, dryer felts, dryer cans, and calendar stacks. The difficulties related to these deposits included direct interference with the efficiency of the contaminated surface, therefore, reduced production, as well as holes, dirt, and other sheet defects that reduce the quality and usefulness of the paper for operations that follow like coating, converting or printing.

From a physical standpoint, “stickies” have usually been particles of visible or nearly visible size in the stock which originate from the recycled fiber. These deposits tend to accumulate on many of the same surfaces that “pitch” can be found on and causes many of the same difficulties that “pitch” can cause. The most severe “stickies” related deposits, however, tend to be found on paper machine wires, wet felts, dryer felts and dryer cans.

Methods of preventing the build-up of deposits on the pulp and paper mill equipment and surfaces are of great importance to the industry. The paper machines could be shut down for cleaning, but ceasing operation for cleaning is undesirable because of the consequential loss of productivity, yet poor paper quality results from the contamination and “dirt” that occurs when deposits break off and become incorporated into the paper sheet. Preventing deposition is thus greatly preferred where it can be effectively practiced.

In the past stickies deposits and pitch deposits have typically manifested themselves in different systems. This was true because mills usually used only virgin fiber or only recycled fiber. Often very different treatment chemicals and strategies were used to control these separate problems.

Current trends are for increased mandatory use of recycled fiber in all systems. This is resulting in a co-occurrence of stickies and pitch problems in a given mill.

Nonionic polymeric detackifier, a material that controls pitch and stickies deposition, used to control pitch and stickies deposition in pulp and papermaking systems is known to those skilled in the art. Poly[vinyl alcohol-co-vinyl acetate] is taught to be effective in controlling the deposition of pitch and stickies contaminants from pulp and papermaking systems in U.S. Pat. No. 4,871,424 and U.S. Pat. No. 4,856,575, respectively. In European Pat. No. EP 0 568 229 A1 hydrophobically modified nonionic associative polymers such as hydrophobically modified hydroxyethyl cellulose ether (HMHEC) are taught to be effective in inhibiting the deposition of organic contaminants from pulp in pulp and papermaking systems. Combinations of nonionic polymers poly[vinyl alcohol-co-vinyl acetate] and HMHEC with cationic polymers are disclosed in U.S. Pat. No. 5,723,021 and U.S. Pat. No. 7,166,192.

Enzymes also are known to be effective as contaminant control agents in pulp and papermaking systems. Use of lipase to hydrolyze the non-polar triglyceride constituent of pitch to water-soluble glycerol and polar fatty acid in the production of mechanical pulp, or mechanical pulp containing paper, is taught in U.S. Pat. No. 5,170,790. Use of a lipolytic enzyme to hydrolyze polymers comprising vinyl acetate to reduce the tackiness of the contaminant, typical of those found as a constituent of stickies contaminant in recycle paper, is taught in PCT publication WO 02/095127 A2.

The use of enzymes alone may not be an optimum method to control organic contaminants in pulp and papermaking applications. For example, the fatty acids resulting from use of a lipase to control pitch can themselves manifest as a deposit on the processing equipment and/or on the final product. U.S. Pat. Nos. 5,256,252 and 5,667,634 teaches a method of controlling pitch deposits in a pulp and papermaking process comprising the use of a combination of a lipase and a cationic polymer to reduce the fatty acid concentration in the aqueous phase of the cellulosic slurry. A similar approach employing a combination of at least one esterase and a cationic polymer to control organic contaminants in recycled paper is disclosed in U.S. Pat. No. 6,471,826 B2. U.S. Pat. Appl. Pub. No. 2004/0194903 A1 discloses a method for reducing or inhibiting the deposition of contaminants on or within press felts comprising one or more enzymes and a non-enzymatic liquid felt conditioner consisting of one or more surfactants and/or one or more anionic or cationic dispersants or polymers. A method to enhance removal of or control adhesives and sticky contaminants in paper processing comprising a combination of one or more enzymes and one or more absorbents or adsorbents is disclosed in U.S. Pat. Appl. Pub. No. 2006/0048908 A1. Said absorbents and adsorbents are selected from the group of natural or synthetic inorganic and organic particles including cross-linked cationic, anionic, or nonionic organic micro particles.

### SUMMARY OF THE INVENTION

The present invention provides for compositions and methods for inhibiting the depositions of organic contaminants from pulp and papermaking systems. The methods comprise adding to the pulp or applying to the surfaces of papermaking machinery an effective deposition inhibiting amount of a combination of an enzyme and a non-ionic polymeric detackifier.

### DETAILED DESCRIPTION OF THE INVENTION

The present invention discloses compositions and methods for inhibiting the deposition of organic contaminants from pulp on the surface of papermaking machinery in pulp and papermaking systems comprising adding to pulp or applying to the surfaces of the paper making machinery an effective deposition inhibiting amount of a combination of components comprising an enzyme and a nonionic polymeric detackifier. The present invention provides for methods for inhibiting the deposition of organic contaminants, such as pitch and stickies, from pulp and papermaking systems.

The term “papermaking systems” is meant to include all pulp processes. This may include but not limited to Kraft, acid sulfite, mechanical pulp and recycled fiber systems. For example, deposition in the brown stock washer, screen room and decker system in Kraft papermaking processes. The phrase “enzyme and nonionic polymeric detackifier combination” is meant to include either combined (if the components are compatible) or separate feeds of the components at either the same or different stages in the papermaking system.

Organic contaminants include constituents which occur in the pulp (virgin, recycled or combinations thereof) having the potential to deposit and reduce paper machine performance or paper quality. These contaminants include, but are not limited to, natural resins such as fatty acids, resin acids, their insoluble salts, fatty esters, sterols; and other organic constituents such as ethylene bis-stearamide, waxes, sizing agents, adhesives, hot melts, inks, defoamers, and latexes which may deposit in papermaking systems.

One of the components used in the present invention is a nonionic polymeric detackifier. Examples of nonionic polymeric detackifier include, but are not limited to, poly[vinyl alcohol-co-vinyl acetate] (PVA/A) and hydrophobically modified hydroxyethyl cellulose ether (HMHEC).

HMHEC is a general descriptor of a family of chemical compounds that are based on hydroxyethyl cellulose (HEC) substrate and differ by what n-alkyl moieties are attached, the amount of hydrophobes, as well as the type of linkage between the cellulose substrate and the attached moiety. HMHEC is usually prepared from HEC by chemically incorporating a hydrophobic n-alkyl moiety generally having from 2 to more than 20 carbon atoms, onto the HEC. The hydrophobe can be linear or branched and is typically attached via an ester or ether linkage. The amount of hydrophobe incorporated will be dependent upon the intended use. The chemical and physical characteristics of HMHEC are determined by the number of carbon atoms in the hydrophobe, amount of hydrophobes, as well as the type of linkage that connects the hydrophobe to the HEC substrate.

The compositions disclosed in U.S. Pat. Nos. 4,228,277 and 6,054,511 are illustrative of HMHEC compounds. In one embodiment of the invention the preferred HMHEC is comprised of an ether linkage and a nominal C₁₆ hydrophobe. An example of a HMHEC of the present invention is DETAC® DC7225 (Hercules Incorporated, Wilmington, Del., USA).

PVA/A is a general descriptor of a family of polymeric compounds based on having hydroxyl groups pendant to the polymer backbone, and that also contain some hydrophobic groupings such as acetate, propionate, butyrate, oleate and the like, but should not contain so much hydrophobic grouping as to render the polymeric material water insoluble. The PVA/A polymeric materials can have molecular weight ranges from about 1,000 to 250,000 or greater. These compounds are typically prepared from polymers or copolymers which yield the hydroxyl group on hydrolysis. The PVA/A which have been found most suitable in accordance to the present invention are those derived from poly[vinyl acetate] which have been from about 50% to 100% hydrolyzed.

The compositions disclosed in U.S. Pat. No. 4,871,424 are illustrative of the PVA/A compounds. In one embodiment of the invention the preferred PVA/A is derived from poly[vinyl acetate] with a nominal molecular weight of 100,000 and from about 80% of the acetate groups have been hydrolyzed to hydroxyl groups. An example of a PVA/A of the present invention is DETAC® DC3970 (Hercules Incorporated, Wilmington, Del., USA).

Enzyme is a generic descriptor for a class of protein catalyst which can promote hydrolysis of triglycerides found as a component in pitch, interact with stickies to render them less tacky, and/or remove and/or inhibit deposition of substances on or in a press felt. Exemplary enzymes include, but are not limited to, the following: amylases, cellulases, cutinases, endoglucanases, esterase, hemicellulases, glucosidases, β-glucose oxidases, laccases, lipases, pectinases, pectate lyases, peroxidases, proteases, pullulanases, and lipolytic enzyme capable of hydrolyzing polymers comprising the vinyl acetate monomer.

The enzymes disclosed in U.S. Pat. Nos. 5,507,952, 5,356,800, 6,471,826 B2, U.S. Pub. No. 2006/0048908, and World International Intellectual Property Organization Pub. Num. WO 02/095127 A2 are illustrative of enzymes of this invention. In one preferred embodiment of the invention the enzyme in a lipase. An example of a commercial lipase of the present invention is RESINASE® A 2× (Novozymes A/S, Bagsvaerd, Denmark).

The enzyme and nonionic polymeric detackifier combination of the present invention is used in an amount effective to inhibit the deposition of organic contaminants such as pitch and stickies. The amount and ratio of enzyme and nonionic polymeric detackifier useful in the present invention varies depending on the source of the cellulosic fiber, operational parameters of the papermaking system, and the activity of the enzyme. The amount of enzyme and nonionic polymeric detackifier typically can range from about 0.1 to 10,000 ppm per ton of pulp on a dry pulp basis.

In one embodiment of the invention the cellulosic slurry to be treated is at an elevated temperature at the time the enzyme and nonionic polymeric detackifier combination of the present invention are added to the pulp and papermaking systems. In general, the temperature of the cellulosic slurry is preferably from about 25° C. to about 120° C. The pH of the cellulosic slurry may be in a range of 3.5 to 12.0. It is known to those skilled in the art that selection of the enzyme and nonionic polymeric detackifier combination application point must take the operational parameters of the pulp and papermaking system into account. For example, certain enzymes are known to denature at high temperatures and pH extremes. For an enzyme it may be preferred that the temperature and pH of the cellulosic slurry range from about 25° C. to 90° C. and from about pH 4.5 to 9.5, respectively; whereas the nonionic polymeric detackifier, for example an ether linkage HMHEC, can function outside these operational constraints. Thus, in some pulp and papermaking systems it may be preferred to add the enzyme and nonionic polymeric detackifier combination components separately at different stages in the system based on operational parameters such as temperature, pH, oxidation potential, residence time, and the like.

The enzyme and nonionic polymeric detackifier combination of the present invention are effective at inhibiting the deposition of organic contaminants in papermaking systems. Generally, it is thought that the compositions of the present invention can be utilized to inhibit deposition on all surfaces of the papermaking system from the pulp mill to the reel of the paper or pulp machine under a variety of system conditions. More specifically, the enzyme and nonionic polymeric detackifier combination of the present invention can effectively decrease the deposition not only on metal surfaces but also on plastic and synthetic surfaces such as machine wires, felts, foils, Uhle boxes, rolls and head box components.

The enzyme and nonionic polymeric detackifier combination of the present invention may be compatible with other pulp and papermaking additives or mixtures thereof. These can include, but are not limited to, starches; fillers such as titanium dioxide; defoamers; wet strength resins; cationic polymers; anionic polymers; and sizing aids.

The enzyme and nonionic polymeric detackifier combination of the present invention can be added to the papermaking system at any stage. They may be added directly to the pulp furnish or indirectly to the furnish through the head box. The enzyme and nonionic polymeric detackifier combination of the present invention may also be applied to surfaces that can suffer from deposition, such as the wire, press felts, press rolls and other deposition-prone surfaces. Application onto the surfaces can be by means of spraying or by any other means that coats the surfaces.

The enzyme and nonionic polymeric detackifier combination of the present invention can be fed concurrently at the same stage in the papermaking system, or separately at different stages in the papermaking system. In one embodiment of the invention one or more enzymes, and one or more nonionic polymeric detackifier, can be added to the same or separate stages in the papermaking system. The enzyme and nonionic polymeric detackifier combination can also be blended together as a single feed of a formulated provided the choices of materials are compatible with each other.

The enzyme and nonionic polymeric detackifier combination of the present invention can be added to the papermaking system neat as a powder, a dispersion in an aqueous salt solution, a solution or dispersion in conjunction with a surfactant, or a solution, the preferred primary solvent being water but is not limited to such. Commercial liquid enzyme and nonionic polymeric detackifier often contain, in addition to the active component, various diluents and/or preservatives designed to stabilize the product and/or settling within the liquid. Such materials include, but are not limited to, propylene glycol, ethoxylated fatty alcohol surfactants, sorbitol, glycerol, sucrose, maltodextrin, calcium salts, sodium chloride, boric acid, potassium sorbate, methionion, and benzisothiazolinone. These materials as well as other known formulation aids such as defoamers, viscosity modifiers, and pH adjuncts such as alkanolamines can additionally be present in the enzyme and nonionic polymeric detackifier combination of the present invention.

When added by spraying techniques, the enzyme and nonionic polymeric detackifier combination is preferably diluted with water or other solvent to a satisfactory inhibitor concentration. The enzyme and nonionic polymeric detackifier combination of the present invention may be added specifically and only to a furnish identified as contaminated or may be added to blended pulps. The enzyme and nonionic polymeric detackifier combination of the present invention may be added to the stock at any point prior to the manifestation of the deposition problem and at more than one site when more than one deposition site occurs. Combinations of the above additive methods may also be employed by feeding the enzyme and nonionic polymeric detackifier, by way of feeding the pulp mill stock, feeding to the paper machine furnish, and/or spraying on the wire and the felt simultaneously.

The combination of components comprising an enzyme and a nonionic polymeric detackifier of the present invention have proven effective against both the pitch and stickies manifestation of organic deposition problems providing for an effective reduction of these problems in paper mills utilizing a variety of virgin and recycled fiber sources.

The present invention will now be further described with reference to a number of specific examples that are to be regarded solely as illustrative and not restricting the scope of the present invention.

### EXAMPLES

### Pitch Deposition Test (PDT)

The Pitch Deposition Test (PDT) was conducted in order to establish the efficacy of the Inventive compositions as deposition control agents. In this test, to a 0.5% consistency bleached hardwood Kraft pulp in DI (deionized) water at approximately 50° C. was added 6 ml of a 25 wt % solution of calcium chloride dihydrate and 140 ml of a synthetic pitch which was preheated to approximately 50° C. The synthetic pitch was prepared according the following procedure: 4.0 g of Wesson Brand Corn Oil (ConAgra Foods, Inc., Omaha, Nebr., USA) and 1.0 g Sylvatol 40 (Arizona Chemical, Jacksonville, Fla., USA) were mixed together and then charged to 995.0 g DI water warm to approximately 50° C. and mixed with a Silverson L4RT lab mixer equipped with an emulsifier screen for two minutes. After addition of the synthetic pitch, an aliquot of the test solution was transferred to an appropriate beaker and mixing initiated via a magnetic stirrer. After equilibrating for 20-minutes, the treatment and two test slides comprised of 3M SCOTCH® Box Sealing Tape (SCOTCH® 375) mounted to 35 mm film slide mounts and suspended in the solution by a fixed holder. After mixing at approximately 50° C. for 2-hours, the slides were removed from the solution, rinsed with 50° C. DI water, double rinsed with RT DI water, and then air dried at 50° C. for 1-hour. The reduction in pitch deposition was determined by taking the average of eight UV absorption measurements at 240 nm and comparing the reduction in absorbance relative to a blank. In this test the adhesive layer of the tape served as a proxy for stickies contamination, while the polypropylene backing as a substrate for pitch deposition. This resulted in the reading being an evaluation of the combination of stickies detackification and pitch deposition.

The results of the testing are summarized in Table 1. The level of RESINASE® A 2× in the examples is reported as ppm on a dry pulp basis as product. The level of DETAC® DC3970 and DETAC® DC7225 in the examples is reported as ppm on a dry pulp basis as nonionic polymeric detackifier active.

TABLE 1

| Example | Treatment | ppm | Absorbance | PDT |
|---|---|---|---|---|
| 1-1 | None | — | 1.038 | 0% |
| 1-2 | RESINASE ® A 2X | 250 | 0.637 | 39% |
| 1-3 | RESINASE ® A 2X | 250 | 0.259 | 75% |
| | DETAC ® DC3970 | 2 | | |
| 1-4 | RESINASE ® A 2X | 250 | 0.403 | 61% |
| | DETAC ® DC7225 | 2 | | |
| 2-1 | None | — | 1.528 | 0% |
| 2-2 | RESINASE ® A 2X | 250 | 0.992 | 54% |
| 2-3 | DETAC ® DC3970 | 2 | 1.419 | 7% |
| 2-4 | RESINASE ® A 2X | 250 | 0.299 | 80% |
| | DETAC ® DC3970 | 2 | | |
| 3-1 | None | — | 1.066 | 0% |
| 3-2 | RESINASE ® A 2X | 250 | 1.175 | −10% |
| 3-3 | DETAC ® DC7225 | 2 | 0.962 | 10% |
| 3-4 | RESINASE ® A 2X | 250 | 0.756 | 29% |
| | DETAC ® DC7225 | 2 | | |

The results presented in sample set Examples 1-1 through 1-4 of Table 1 demonstrate that significant improvements in inhibiting the deposition of organic contaminants resulted when employing the enzyme and nonionic polymeric detackifier combination of the present invention versus use of the enzyme alone. The results presented in sample sets 2-1 through 2-4 and 3-1 through 3-4 of Table 1 demonstrate that use of the enzyme and nonionic polymeric detackifier combination of the present invention outperformed the use of either the enzyme or the nonionic polymeric detackifier as a standalone treatment.

While the present invention has been described with respect to a particular embodiment, it is apparent that numerous other forms and modifications will be obvious to those skilled in the art. The appended claims and this invention generally should be construed to cover all such obvious forms and modifications that are within the true scope of the present invention.

1. A method for inhibiting the deposition of one or more organic contaminants in pulp and papermaking systems comprising treating one or both of the pulp or of the process equipment surfaces in a pulp and papermaking system with an effective inhibiting amount of a combination of 1) one or more enzymes and 2) one or more nonionic polymeric detackifiers.

2. The method of claim 1 wherein the one or more enzymes are selected from the group consisting of amylases, cellulases, cutinases, endoglucanases, esterase, hemicellulases, glucosidases, β-glucose oxidases, laccases, lipases, pectinases, pectate lyases, peroxidases, proteases, pullulanases, and lipolytic enzymes.

3. The method of claim 1 wherein the one or more enzymes comprise a lipase.

4. The method of claim 1 wherein the one or more nonionic polymeric detackifiers comprise hydrophobically modified hydroxyethyl cellulose ether.

5. The method of claim 1 wherein the one or more nonionic polymeric detackifiers comprise a poly[vinyl acetate] having from about 50% to 100% hydrolysis of the acetate groups to hydroxyl groups.

6. The poly[vinyl acetate] of claim 5 wherein the hydrolysis of acetate groups to hydroxyl groups is about 80% or more.

7. The method according to claim 1 wherein the one or more enzymes are added in combination with one or more nonionic polymeric detackifiers at multiple stages of a pulp and papermaking system.

8. The method of claim 7 wherein the one or more enzymes and the one or more nonionic polymeric detackifiers are added separately to different stages of the pulp and papermaking system.

9. The method of claim 7 wherein the one or more enzymes and the one or more nonionic polymeric detackifiers are added to the same stage of the pulp and papermaking system.

10. The method of claim 1 wherein the combination of one or more enzymes and one or more nonionic polymeric detackifiers are sprayed on the process equipment surfaces.

11. The combination of claim 10 wherein the enzyme is a lipase and the nonionic polymeric detackifier is a poly[vinyl acetate] having a nominal molecular weight of 100,000 and about 80% of the acetate groups hydrolyzed to hydroxyl groups.

12. The combination of claim 10 wherein the enzyme is a lipase and the nonionic polymeric detackifier is a hydrophobically modified hydroxyethyl cellulose ether comprising an ether linkage and a nominal C₁₆ hydrophobe.

13. The method of claim 1 comprising directly or indirectly treating the pulp with an effective inhibiting amount of a combination of a lipase and a poly[vinyl acetate] having a nominal molecular weight of 100,000 and about 80% or more of the acetate groups hydrolyzed to hydroxyl groups.

14. The method of claim 1 comprising directly or indirectly treating the pulp with an effective inhibiting amount of a combination of a lipase and a hydrophobically modified hydroxyethyl cellulose ether comprising an ether linkage and a nominal C₁₆ hydrophobe.

15. A composition for inhibiting the deposition of organic contaminants on process equipment surfaces in pulp and papermaking systems comprising one or more enzymes and one or more nonionic polymeric detackifiers.

16. The composition of claim 15 wherein the one or more enzymes comprise a lipase.

17. The composition of claim 15 wherein the one or more nonionic polymeric detackifiers comprise hydrophobically modified hydroxyethyl cellulose ether.

18. The composition of claim 15 wherein the one or more nonionic polymeric detackifiers comprise hydrophobically modified hydroxyethyl cellulose ether comprising an ether linkage and a nominal C₁₆ hydrophobe.

19. The composition of claim 15 wherein the one or more nonionic polymeric detackifiers comprise a poly[vinyl acetate] having from about 50% to 100% hydrolysis of the acetate groups to hydroxyl groups.

20. The composition of claim 15 wherein the enzyme consists essentially of a lipase and the nonionic polymeric detackifiers consist essentially of at least one of a poly[vinyl acetate] having a nominal molecular weight of 100,000 and about 80% or more of the acetate groups hydrolyzed to hydroxyl groups and a hydrophobically modified hydroxyethyl cellulose ether comprising an ether linkage and a nominal C₁₆ hydrophobe.
